(19) United States
(12) Plant Patent Application Publication
(10) Pub. No.: US 2017/0020047 P1
(43) Pub. Date: Jan. 19, 2017

(54) Fuchsia plant named 'KLEFU14843'

(50) ;

(71) Applicant: Klemm+Sohn GmbH & Co. KG, Stuttgart (DE)

(72) Inventors: Nils Klemm, Stuttgart (DE); Andrea Dohm, Pforzheim (DE)

(73) Assignee: Klemm+Sohn GmbH & Co. KG, Stuttgart (DE)

(21) Appl. No.: 14/545,972

(22) Filed: Jul. 13, 2015

(51) Int. Cl. A01H 5/00

(52) U.S. Cl. PLT/300

(57) ABSTRACT

A new fuchsia plant particularly distinguished by flowers having white petals and red-purple sepals, a compact, mounded, upright plant habit, and a freely-branching growth habit, is disclosed.

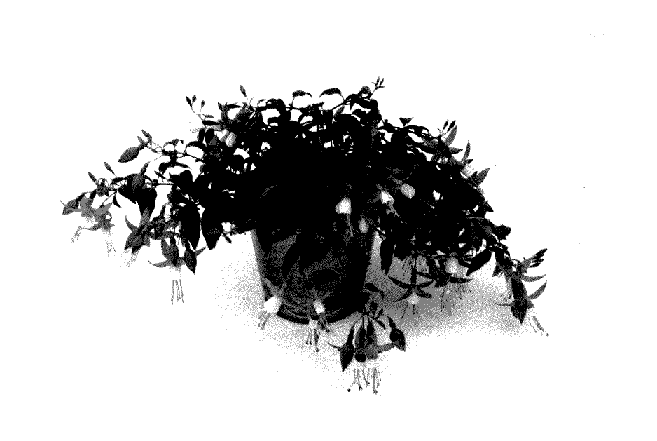

### GENUS AND SPECIES

Fuchsia sp.

### VARIETY DENOMINATION

‘KLEFU 14843’

### BACKGROUND OF THE NEW PLANT

The present invention comprises a new and distinct variety of fuchsia, botanically known as Fuchsia sp. and hereinafter referred to by the variety name ‘KLEFU14843’. ‘KLEFU14843’ originated from a controlled-cross in October 2011 between the proprietary female fuchsia variety ‘2012.15’ (unpatented) and the proprietary male fuchsia variety ‘2012.7’ (unpatented).

In May 2012, the two fuchsia parental lines ‘2012.15’ and ‘2012.7’ were crossed in Stuttgart, Germany and seeds were obtained. The seeds were sown and plants were grown for evaluation. A plant line was selected in March 2013 and subsequently named ‘KLEFU14843’. In March 2013, ‘KLEFU 14843’ was first vegetatively propagated by vegetative cuttings in Stuttgart, Germany. ‘KLEFU14843’ was found to reproduce true to type in successive generations of asexual propagation via vegetative cuttings in Stuttgart, Germany.

### SUMMARY

The following are the most outstanding and distinguishing characteristics of this new variety when grown under normal horticultural practices in Stuttgart, Germany.
1. Flowers having white petals and red-purple sepals;
2. A compact, mounded, upright plant habit; and
3. A freely-branching growth habit.

### DESCRIPTION OF THE PHOTOGRAPH

This new fuchsia plant is illustrated by the accompanying photograph which shows an overall view of the plant. The photograph is of an approximately 3-month-old plant grown in Stuttgart, Germany under greenhouse conditions in May 2014. The colors shown are as true as can be reasonably obtained by conventional photographic procedures.

### DESCRIPTION OF THE NEW VARIETY

The following detailed descriptions set forth the distinctive characteristics of ‘KLEFU14843’. The data which define these characteristics were collected from asexual reproductions carried out in Latina, Italy. Data was collected on 2.5 to 3 month-old plants in Latina, Italy grown in a greenhouse in June 2015. Color references are to The R.H.S. Colour Chart of The Royal Horticultural Society of London (R.H.S.), 5^{th} edition (2007).
Classification:
   Family.—Onagraceae.
   Botanical.—Fuchsia sp.
   Designation.—‘KLEFU 14843’.
Parentage:
   Female parent.—The proprietary female fuchsia variety ‘2012.15’ (unpatented).
   Male parent.—The proprietary male fuchsia variety ‘2012.7’ (unpatented).
Propagation:
   Type.—Vegetative cuttings.
   Time to initiate roots, summer.—About 1 to 2 weeks at 20 ° C. to 25 ° C.
   Time to initiate roots, winter.—About 2 weeks at temperatures of 18 ° C. to 22 ° C.
   Time to produce a rooted young plant, summer.—3 to 4 weeks at temperatures of 20 ° C. to 25 ° C.
   Time to produce a rooted young plant, winter.—4 weeks at temperatures of 18 ° C. to 22 ° C.
   Root description.—Fine, fibrous, yellowish-white in color.
   Rooting habit.—Freely branching; medium density.
Plant:
   Plant habit/form.—Compact, mounded, upright plant habit.
   Growth habit.—Freely-branching habit with lateral branches developing potentially at every node; bushy plant habit; vigorous growth habit.
   Plant height.—20.0 cm to 22.0 cm.
   Plant diameter.—28.0 cm to 30.0 cm.
Lateral branches:
   Length.—Approximately 15.0 cm to 18.0 cm.
   Diameter.—Approximately 2.0 mm.
   Internode length.—Approximately 2.8 cm.
   Strength.—Strong, flexible.
   Aspect.—Initially upright to semi-upright. Texture.—Pubescent. Color.—Close to RHS 146B.
Leaves:
   Arrangement.—Opposite, simple.
   Length.—4.7 cm on average.
   Width.—1.5 cm on average.
   Shape.—Ovate.
   Apex.—Broadly acute.
   Base.—Obtuse and rounded.
   Margin.—Shallowly serrate.
   Texture (both upper and lower surfaces).—Glabrous.
   Venation pattern.—Pinnate; arcuate.
   Venation color.—Upper surface: Close to RHS 146B Lower surface: Close to RHS 147C.
   Color.—Developing leaves: Upper surface: RHS 137A Lower surface: RHS 147B Fully expanded leaves: Upper surface: RHS N137A Lower surface: RHS 147B.
Petiole:
   Length.—Approximately 1.5 cm.
   Diameter.—Approximately 1.5 mm.
   Texture.—Upper surface: Smooth, glabrous, concave Lower surface: Smooth, glabrous, convex.
   Color.—Upper surface: RHS 146B Lower surface: RHS 146C.
Flower:
   Flower arrangement and habit.—Single axillary flowers; flowers pendulous; freely flowering habit with potentially two flowers per leaf axil.
   Total number of flowers per plant.—Over 40 fully open flowers and 80 buds on a mature. plant when at flowering stage.
   Fragrance.—None detected.
   Natural flowering season.—Long flowering period; in Europe, plants flower from early spring to autumn; flowering continuous during this period.
   Flower longevity.—Flowers last about four to six days on the plant.
   Flower diameter.—0.9 cm (petals), 4.4 cm (sepals).
   Flower height (depth).—Approximately 3.5 cm (base of sepals to stigma tip).
   Flower buds.—Shape: Elliptic Length: Approximately 2.5 cm Diameter: Approximately 0.9 cm Color: RHS 51A.
Petals:
   Arrangement.—Typically four in a single whorl.
   Length.—Approximately 1.5 cm.
   Width.—Approximately 1.1 cm.
   Shape.—Obovate.
   Apex.—Obtuse.
   Base.—Cuneate.
   Margin.—Entire.
   Texture (both upper and lower surfaces).—Smooth, glabrous, satiny.
   Color.—Upper (outside) surface: When opening: RHS 155B Fully open: RHS 155B Lower (inside) surface: When opening: RHS 155B Fully open: RHS 155B.
Sepals:
   Arrangement.—Calyx star-shaped with four sepals fused at the base.
   Length.—2.6 cm.
   Width.—0.7 cm.
   Shape.—Elliptic.
   Apex.—Slightly cuspidate.
   Base.—Attenuate, fused.
   Margin.—Entire.
   Texture (both upper and lower surfaces).—Smooth, glabrous, and satiny.
   Color.—Upper (outside) surface: When opening: RHS 58B Fully opened: RHS 58B Lower (inside) surface: When opening: RHS 58B Fully opened: RHS 58C.
Peduncles:
   Length.—2.6 cm on average.
   Diameter.—0.09 cm.
   Angle.—Arching.
   Strength.—Moderately strong.
   Texture.—Sparsely pubescent.
   Color.—RHS 146B.
Reproductive organs:
   Stamens.—Quantity per flower: 8 Anther shape: Ellipsoidal Anther size: About 2.2 mm by 1.3 mm Anther color: RHS 158B with RHS N57B (Red-Purple) shading Pollen amount: Scarce Pollen color: Close to RHS 155A.
   Pistils.—Quantity: 1 per flower Pistil length: 4.5 cm on average Style color: Towards the base, close to RHS 155A; towards the apex, close to RHS 54A Stigma shape: Clavate Stigma color: RHS 49B Ovary color: RHS 144A.
Fruit and seed set: None observed
Disease and pest/insect resistance: None observed

### COMPARISON WITH PARENTAL LINES AND KNOWN VARIETY

‘KLEFU 14843’ is distinguished from its parents as described in Table 1:

TABLE 1
Comparison with Parental Lines

| | | Female parent | Male parent |
|---|---|---|---|
| Trait | ‘KLEFU14843’ | ‘2012.15’ | ‘2012.7’ |
| Plant habit | Mounded | Semi-mounded | Semi-mounded |

‘KLEFU 14843’ can be compared to the commercial variety WINDCHIMES ‘Red White’ (patent status unknown). Differences between the two varieties are described in Table 2:

TABLE 2
Comparison with Similar Variety

| | | Commercial line |
|---|---|---|
| Trait | ‘KLEFU14843’ | WINDCHIMES ‘Red White’ |
| Sepal color | Light red | Dark red |

We claim:

1. A new and distinct variety of fuchsia plant named ‘KLEFU 14843’ as illustrated and described herein.
